(19) United States
(12) Patent Application Publication
(10) Pub. No.: US 2021/0298328 A1
(43) Pub. Date: Sep. 30, 2021

(54) PET FOOD PRODUCTS COMPRISING OLIGOSACCHARIDES AND METHODS OF USE

(71) Applicants: MARS, INCORPORATED, McLean, VA (US); THE REGENTS OF THE UNIVERSITY OF CALIFORNIA, Oakland, CA (US)

(72) Inventors: Kevin Hughes, Leicestershire (GB); Carlito Lebrilla, Sacramento, CA (US); Paul Glyn Jones, Leicestershire (GB); David J. Wrigglesworth, Leicestershire (GB)

(73) Assignees: MARS, INCOROPRATED, McLean, VA (US); THE REGENTS OF THE UNIVERSITY OF CALIFORNIA, Oakland, CA (US)

(21) Appl. No.: 17/266,316

(22) PCT Filed: Aug. 8, 2019
(86) PCT No.: PCT/US2019/045733

§ 371 Date: Feb. 5, 2021

Related U.S. Application Data
(60) Provisional application No. 62/716,191, filed on Aug. 8, 2018.

(51) Int. Cl. A23K 20/163; A23K 50/40; A23K 10/18; A61K 31/702; A61K 35/745; A61K 35/747; A61K 35/744

(52) U.S. Cl. CPC A23K 20/163; A23K 50/40; A23K 10/18; A61K 35/744; A61K 35/745; A61K 35/747; A61K 31/702

(57) ABSTRACT

The presently disclosed subject matter relates to food products comprising oligosaccharides for an animal, such as a domestic animal or a pet. In certain embodiments, the food product can be a feline food product or a canine food product.

### CROSS-REFERENCE TO RELATED APPLICATION

This application claims priority to U.S. Provisional Application Ser. No. 62/716,191 filed Aug. 8, 2018, the contents of which are incorporated by reference herein in their entirety.

### FIELD OF THE INVENTION

The presently disclosed subject matter relates to food products comprising oligosaccharides for an animal, such as a domestic animal or a pet (e.g., a cat or a dog).

### BACKGROUND OF THE INVENTION

Milk oligosaccharides (MOs) are among the most abundant components of milk besides lactose, proteins and fat. Certain oligosaccharides can feed probiotic microorganisms residing in the gastrointestinal tract, which can reduce the colonization of pathogenic microorganisms and promote a healthy intestinal microbiome. Thus, there is a need for animal food products comprising suitable contents of oligosaccharides to promote animal health and immunity.

### SUMMARY OF THE INVENTION

The presently disclosed subject matter provides for a companion animal food product comprising at least one oligosaccharide. In certain non-limiting embodiments, the at least one oligosaccharide selected from the group consisting of 3′-fusosyllactose (3′FL), difucosyl-para-lacto-N-hexoase II (DFpLNH II), lacto-difucosyl-tetraose (LDFT), difucosyllacto-N-hexaose b (DFLNHb), lacto-N-neohexaose (LNnH), 3′-sialyllactose (3′SL), 2′-fucosyllactose (2′FL), 6′-sialyllactose (6′SL), lacto-N-neotetraose (LNnT), lacto-N-tetraose (LNT), lacto-N-fucopentaose II (LNFP II), fucosyl-paralacto-N-hexaose III (IFLNH III), monofucosyl-paralacto-N-hexaose IV (MFpLNH IV), lacto-N-hexaose (LNH), sialyl-lacto-N-neohexaose II (S-LNnH II), and para-lacto-N-hexaose (pLNH) or any combination thereof. In certain embodiments, the companion animal is a feline or a canine.

The presently disclosed subject matter provides for a companion animal food product comprising oligosaccharides difucosyllacto-N-hexaose b (DFLNHb) and lacto-N-neohexaose (LNnH). In certain embodiments, the companion animal is a feline. In certain embodiments, the companion animal food product further comprises a oligosaccharide selected from the group consisting of 3′-sialyllactose (3′SL), 2′-fucosyllactose (2′FL), 6′-sialyllactose (6′SL), lacto-N-neotetraose (LNnT), lacto-N-tetraose (LNT), lacto-N-fucopentaose II (LNFP II), fucosyl-paralacto-N-hexaose III (IFLNH III), monofucosyl-paralacto-N-hexaose IV (MFpLNH IV), lacto-N-hexaose (LNH), sialyl-lacto-N-neohexaose II (S-LNnH II), 3′-fusosyllactose (3′FL), difucosyl-para-lacto-N-hexoase II (DFpLNH II), lacto-difucosyl-tetraose (LDFT), para-Lacto-N-hexaose (pLNH) and any combination thereof. In certain embodiments, the oligosaccharides further comprise one or more of 3′-sialyllactose (3′SL), 2′-fucosyllactose (2′FL) and lacto-N-neotetraose (LNnT). In certain embodiments, a total concentration of the oligosaccharides is from about 0.3 g/L to about 5.0 g/L in a final food product.

The presently disclosed subject matter provides for a companion animal food product comprising oligosaccharides 2′-fucosyllactose (2′FL) and lacto-N-neotetraose (LNnT). In certain embodiments, the companion animal food product further comprises a oligosaccharide selected from the group consisting of difucosyllacto-N-hexaose b (DFLNHb), lacto-N-neohexaose (LNnH), 3′-sialyllactose (3′SL), 6′-sialyllactose (6′SL), lacto-N-tetraose (LNT), lacto-N-fucopentaose II (LNFP II), fucosyl-paralacto-N-hexaose III (IFLNH III), monofucosyl-paralacto-N-hexaose IV (MFpLNH IV), lacto-N-hexaose (LNH), sialyl-lacto-N-neohexaose II (S-LNnH II), 3′-fusosyllactose (3′FL), difucosyl-para-lacto-N-hexoase II (DFpLNH II), lacto-difucosyl-tetraose (LDFT), para-lacto-N-hexaose (pLNH) and any combination thereof.

The presently disclosed subject matter provides for a companion animal food product comprising oligosaccharides 3′-sialyllactose (3′SL), 2′-fucosyllactose (2′FL) and 6′-sialyllactose (6′SL), wherein the companion animal is a canine and wherein a total concentration of the oligosaccharides is from about 0.3 g/L to about 5.0 g/L in a final food product. In certain embodiments, the total concentration of the oligosaccharides is from about 1.2 g/L to about 2.2 g/L in a final food product.

The presently disclosed subject matter provides for a companion animal food product comprising oligosaccharides difucosyllacto-N-hexaose b (DFLNHb), 3′-sialyllactose (3′SL) and lacto-N-neohexaose (LNnH), wherein a total concentration of the oligosaccharides is from about 0.3 g/L to about 5.0 g/L in a final food product. In certain embodiments, the DFLNHb, 3′SL and LNnH are in a ratio by weight of about 8 to about 6 to about 5, respectively. In certain embodiments, the companion animal is a feline.

The presently disclosed subject matter provides for a companion animal food product comprising oligosaccharides 3′-sialyllactose (3′SL), lacto-N-neotetraose (LNnT), 2′-fucosyllactose (2′FL) and lacto-N-tetraose (LNT), wherein a total concentration of the oligosaccharides is from about 0.5 g/L to about 1.5 g/L in a final food product. In certain embodiments, the 3′SL, LNnT, 2′FL and LNT are in a ratio by weight of about 6 to about 3 to about 0.6 to about 0.1, respectively. In certain embodiments, the companion animal is a feline or a canine.

The presently disclosed subject matter provides for a companion animal food product comprising oligosaccharides 3′-sialyllactose (3′SL), 2′-fucosyllactose (2′FL), 6′-sialyllactose (6′SL) and lacto-N-neotetraose (LNnT), wherein a total concentration of the oligosaccharides is from about 0.01 g/L to about 1.5 g/L in a final food product. In certain embodiments, the 3′SL, 2′FL, 6′SL and LNnT is in a ratio by weight of about 46 to about 23 to about 17 to about 0.4, respectively. In certain embodiments, the companion animal is a canine.

In certain embodiments, the companion animal food product further comprises a probiotic.

The presently disclosed subject matter provides for a companion animal food product comprising: a) at least one oligosaccharides selected from the group consisting of difucosyllacto-N-hexaose b (DFLNHb), lacto-N-neohexaose (LNnH), 3′-sialyllactose (3′SL), 2′-fucosyllactose (2′FL), 6′-sialyllactose (6′SL), lacto-N-neotetraose (LNnT), lacto-N-tetraose (LNT), lacto-N-fucopentaose II (LNFP II), fucosyl-paralacto-N-hexaose III (IFLNH III), monofucosyl-paralacto-N-hexaose IV (MFpLNH IV), lacto-N-hexaose (LNH), sialyl-lacto-N-neohexaose II (S-LNnH II), 3′-fusosyllactose (3′FL), difucosyl-para-lacto-N-hexoase II (DFpLNH II), lacto-difucosyl-tetraose (LDFT), and para-lacto-N-hexaose (pLNH); and b) a probiotic. In certain embodiments, the companion animal food product comprises at least two oligosaccharides. In certain embodiments, the companion animal food product comprises at least three oligosaccharides. In certain embodiments, the companion animal is a feline or a canine.

In certain embodiments, the probiotic is a feline probiotic. In certain embodiments, the probiotic is a canine probiotic. In certain embodiments, the probiotic is selected from the group consisting of any organism from lactic acid bacteria and more specifically from the following bacterial genera; Lactococcus spp., Pediococcus spp., Bifidobacterium spp. (e.g., B. longum B. bifidum, B. pseudolongum, B. animalis), Lactobacillus spp. (e.g., L. bulgaricus, L. acidophilus, L. brevis, L casei, L. rhamnosus, L. plantarum, L. reuteri, L. fermentum, Enterococcus spp. (e.g. E. faecium), Prevotella spp., Fusobacteria spp, Alloprevotella spp, Bacteroides spp., and any combination thereof.

In certain embodiments, the companion animal food product further comprises a food additive. In certain embodiments, the food additive is selected from a flavor composition, a stabilizer, a thickener and any combination thereof.

In certain embodiments, the companion animal food product comprises 5-40% protein, 5-45% fat, and 15-25% carbohydrate.

In certain embodiments, the companion animal is less than about 52 days old. In certain embodiments, the companion animal is less than about 40 days old. In certain embodiments, the companion animal is a canine and is less than about 38 days old. In certain embodiments, the companion animal is a feline and is less than about 36 days old. In certain embodiments, the companion animal is less than about 10 days old. In certain embodiments, the companion animal is less than about 4 days old.

In certain embodiments, the concentration of at least one oligosaccharide decreases over the course of feeding a companion animal. In certain embodiments, the concentration of at least one oligosaccharide increases over the course of feeding a companion animal. In certain embodiments, the concentration of at least one oligosaccharide is modified based on the age of the companion animal.

In certain embodiments, the companion animal food product is a food supplement. In certain embodiments, the companion animal food product is in a form of powder, tablet or liquid.

The presently disclosed subject matter provides for use of a companion animal food product of any one of the preceding claims for improving immunity, digestive function and/or decreasing inflammation.

The presently disclosed subject matter provides for a method for improving immunity, digestive function and/or decreasing inflammation of a companion animal comprising administering an effective amount of a companion animal food product disclosed herein to a companion animal in need thereof.

The presently disclosed subject matter provides for a method for increasing a probiotic in a companion animal comprising administering an effective amount of a companion animal food product disclosed herein to a companion animal in need thereof.

The presently disclosed subject matter provides for a method for modifying a gastrointestinal (GI) microbiome in a companion animal comprising administering an effective amount of a companion animal food product disclosed herein to a companion animal in need thereof. In certain embodiments, the companion animal is a feline or a canine. In certain embodiments, the companion animal has a digestive disorder and/or inflammation.

The presently disclosed subject matter provides for a method for improving immunity, digestive function and/or decreasing inflammation of a companion animal comprising administering to a companion animal in need thereof oligosaccharides 2′-fucosyllactose (2′FL) and lacto-N-neotetraose (LNnT). In certain embodiments, the 2′FL is at a dosage from about 100 mg/kg body weight per day to about 1000 mg/kg bodyweight per day and LNnT is at a dosage from about 10 mg/kg body weight per day to about 30 mg/kg body weight per day. In certain embodiments, the companion animal is a canine. In certain embodiments, the canine is less than 38 days old.

The presently disclosed subject matter provides for a method for improving immunity, digestive function and/or decreasing inflammation of a companion animal comprising administering to a companion animal in need thereof an oligosaccharide selected from the group consisting of 3′-sialyllactose (3′SL), 2′-fucosyllactose (2′FL), 6′-sialyllactose (6′SL) and any combination thereof. In certain embodiments, the oligosaccharide is at a dosage from about 10 mg/kg body weight per day to about 50 mg/kg body weight per day. In certain embodiments, the companion animal is a canine or a feline.

The presently disclosed subject matter provides for a method of manufacturing a companion animal food product disclosed herein, the method comprising, admixing the oligosaccharides with a protein source, a carbohydrate source, a fat source or any combination thereof, thereby manufacturing the companion animal food product.

### BRIEF DESCRIPTION OF THE DRAWINGS

FIG. 1 depicts an exemplary workflow for determining oligosaccharide contents in milk.

FIG. 2 depicts a comparison of total concentrations of oligosaccharides in human, canine and feline milk.

FIG. 3 depicts the oligosaccharide profile of feline milk.

FIG. 4 depicts the oligosaccharide profile of canine milk.

### DETAILED DESCRIPTION OF THE INVENTION

To date, there remains a need for animal food products comprising suitable contents of oligosaccharides for improving health and/or immunity. The present application relates to companion animal food products comprising one or more oligosaccharides and methods for improving health and/or immunity of a companion animal using such food products. In certain embodiments, the oligosaccharide is present in an animal milk or is similar to, a variant or a derivative of a milk oligosaccharides. In certain embodiments, the oligosaccharides can be consumed by a companion animal or a probiotic of the companion animal and thereby improve the health of the companion animal.

For clarity and not by way of limitation, the detailed description of the presently disclosed subject matter is divided into the following subsections:

1. Definitions;

2. Oligosaccharides;

3. Method of Use; and

4. End products.

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition to improving immunity, digestive function and/or decreasing inflammation, an effective amount of a composition described herein is an amount sufficient to improving immunity, digestive function and/or decreasing inflammation, as well as decrease the symptoms and/or reduce the likelihood of a digestive disorder and/or inflammation. An effective treatment described herein is a treatment sufficient to improving immunity, digestive function and/or decreasing inflammation, as well as decrease the symptoms and/or reduce the likelihood of a digestive disorder and/or inflammation. The decrease can be a 1%, 5%, 10%, 20%, 30%, 40%, 50%, 60%, 70%, 80%, 90%, 95%, 98% or 99% decrease in severity of symptoms of a digestive disorder or inflammation, or the likelihood of a digestive disorder or inflammation. An effective amount can be administered in one or more administrations. A likelihood of an effective treatment described herein is a probability of a treatment being effective, i.e., sufficient to treat or ameliorate a digestive disorder and/or inflammation, as well as decrease the symptoms.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. The decrease can be a 1%, 5%, 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

As used herein, and as well-understood in the art, a “probiotic” is a preparation or composition comprising microorganisms that can provide health benefits when consumed. The microorganisms include, but are not limited to bacteria, fungi, yeasts and archaea. In certain embodiments, the probiotic can modify the microbiome in the GI system to enhance the balance of the microbiome in GI system, e.g., by acting as an inoculum for an increased population of beneficial microbes, and/or by antagonizing growth of deleterious microbes. In certain embodiments, the probiotic is an animal probiotic, e.g., a feline probiotic or a canine probiotic.

As used herein, and as well-understood in the art, a “prebiotic” is a substance or a composition that can induce the growth or activity of one or more beneficial microorganism (e.g., one or more probiotics, e.g., bacteria, fungi, yeasts and archaea). In certain embodiments, the prebiotic can modify the microbiome in the GI system to enhance the balance of the microbiome in GI system. In certain embodiments, the prebiotic is indigestible to an animal. In certain embodiments, the prebiotic can induce the growth or activity of one or more animal probiotics, e.g., a feline probiotic or a canine probiotic.

The term “pet food” or “pet food composition” or “pet food product” or “final pet food product” means a product or composition that is intended for consumption by a companion animal, such as a cat, a dog, a guinea pig, a rabbit, a bird or a horse. For example, but not by way of limitation, the companion animal can be a “domestic” dog, e.g., Canis lupus familiaris. In certain embodiments, the companion animal can be a “domestic” cat such as Felis domesticus. A “pet food” or “pet food composition” or “pet food product” or “final pet food product” includes any food, feed, snack, food supplement, liquid, beverage, treat, toy (chewable and/or consumable toys), meal substitute or meal replacement.

### 2. Oligosaccharides

The presently disclosed subject matter provides a food composition comprising one or more oligosaccharide. In certain embodiments, the one or more oligosaccharide is selected from the oligosaccharides that are present in feline milk, or a variant or derivative thereof. In certain embodiments, the one or more oligosaccharide is selected from the oligosaccharides that are present in canine milk or a variant or derivative thereof. In certain embodiments, the one or more oligosaccharide can be an oligosaccharide that is not present in feline milk or canine milk but can be safely consumed by a feline or a canine. In certain embodiments, the one or more oligosaccharide can be 3′-fusosyllactose (3′FL), difucosyl-para-lacto-N-Hexoase II (DFpLNH II), lacto-difucosyl-tetraose (LDFT), difucosyllacto-N-hexaose b (DFLNHb), lacto-N-neohexaose (LNnH), 3′-sialyllactose (3′ SL), 2′-fucosyllactose (2′FL), 6′-sialyllactose (6′SL), lacto-N-neotetraose (LNnT), lacto-N-tetraose (LNT), lacto-N-fucopentaose II (LNFP II), fucosyl-paralacto-N-hexaose III (IFLNH III), monofucosyl-paralacto-N-hexaose IV (MFpLNH IV), lacto-N-hexaose (LNH), sialyl-lacto-N-neohexaose II (S-LNnH II), or para-lacto-N-hexaose (pLNH).

In certain embodiments, the food composition comprises at least two oligosaccharides disclosed herein. In certain embodiments, the food composition comprises at least three oligosaccharides. In certain embodiments, the food composition comprises at least four, five, six, seven, eight or more oligosaccharides. In certain embodiments, the ratio by weight between any two oligosaccharides in the food composition can be varied from about 0.001:1 to about 1000:1. In certain embodiments, the ratio between any two oligosaccharides in the food composition is from about 0.01:1 to about 100:1, from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between any two oligosaccharides in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between any two oligosaccharides in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof.

In certain embodiments, the food composition comprising one or more oligosaccharide selected from the group consisting of difucosyllacto-N-hexaose b (DFLNHb), lacto-N-neohexaose (LNnH), 3′-sialyllactose (3′SL), 2′-fucosyllactose (2′FL), 6′-sialyllactose (6′SL), lacto-N-neotetraose (LNnT), lacto-N-tetraose (LNT) and any combination thereof. In certain embodiments, the food composition is comprised in a cat food product. In certain embodiments, the food composition is comprised in a dog food product.

In certain embodiments, the food composition comprises DFLNHb and LNnH. In certain embodiments, the ratio by weight between DFLNHb and LNnH in the food composition can be varied from about 0.01:1 to about 100:1. In certain embodiments, the ratio between DFLNHb and LNnH in the food composition is from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between DFLNHb and LNnH in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between DFLNHb and LNnH in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof.

In certain embodiments, the food composition further comprises one or more of LNnT, 3′SL or 2′FL. In certain embodiments, the food composition comprises DFLNHb, LNnH and 3′SL. In certain embodiments, the ratio by weight between DFLNHb and 3′SL in the food composition can be varied from about 0.01:1 to about 100:1. In certain embodiments, the ratio between DFLNHb and 3′SL in the food composition is from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between DFLNHb and 3′SL in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between DFLNHb and 3′SL in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof. In certain embodiments, the DFLNHb, 3′SL and LNnH are in a ratio by weight of about 8 to about 6 to about 5, respectively.

In certain embodiments, the food composition comprises 3′SL, 2′FL and 6′SL. In certain embodiments, the food composition further comprises one or more of LNnT, 3′SL or 2′FL. In certain embodiments, the food composition comprises 3′SL, 2′FL, 6′SL, and LNnT. In certain embodiments, the ratio by weight between 3′SL and 2′FL in the food composition can be varied from about 0.01:1 to about 100:1. In certain embodiments, the ratio between 3′SL and 2′FL in the food composition is from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between 3′SL and 2′FL in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between 3′SL and 2′FL in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof. In certain embodiments, the ratio by weight between 3′SL and 6′SL in the food composition can be varied from about 0.01:1 to about 100:1. In certain embodiments, the ratio between 3′SL and 6′SL in the food composition is from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between 3′SL and 6′SL in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between 3′SL and 6′SL in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof. In certain embodiments, the ratio by weight between 3′SL and LNnT in the food composition can be varied from about 0.001:1 to about 1000:1. In certain embodiments, the ratio between 3′SL and LNnT in the food composition is from about 0.002:1 to about 500:1, from about 0.003:1 to about 300:1, from about 0.005:1 to about 200:1, from about 0.01:1 to about 100:1, from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between 3′SL and LNnT in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between 3′SL and LNnT in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof. In certain embodiments, 3′SL, 2′FL, 6′SL and LNnT is in a ratio by weight of about 46 to about 23 to about 17 to about 0.4, respectively.

In certain embodiments, the food composition comprises 3′SL, LNnT, 2′FL, and LNT. In certain embodiments, the ratio by weight between 3′SL and LNnT in the food composition can be varied from about 0.01:1 to about 100:1. In certain embodiments, the ratio between 3′SL and LNnT in the food composition is from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between 3′SL and LNnT in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between 3′SL and LNnT in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof. In certain embodiments, the ratio by weight between 3′SL and 2′FL in the food composition can be varied from about 0.01:1 to about 100:1. In certain embodiments, the ratio between 3′SL and 2′FL in the food composition is from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between 3′SL and 2′FL in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between 3′SL and 2′FL in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof. In certain embodiments, the ratio by weight between 3′SL and LNT in the food composition can be varied from about 0.001:1 to about 1000:1. In certain embodiments, the ratio between 3′SL and LNT in the food composition is from about 0.002:1 to about 500:1, from about 0.003:1 to about 300:1, from about 0.005:1 to about 200:1, from about 0.01:1 to about 100:1, from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between 3′SL and LNT in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between 3′SL and LNT in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof. In certain embodiments, 3′SL, LNnT, 2′FL and LNT are in a ratio by weight of about 6 to about 3 to about 0.6 to about 0.1, respectively.

In certain embodiments, the food composition comprises 2′FL and LNnT. In certain embodiments, the ratio by weight between 2′FL and LNnT in the food composition can be varied from about 0.01:1 to about 100:1. In certain embodiments, the ratio between 2′FL and LNnT in the food composition is from about 0.02:1 to about 50:1, from about 0.05:1 to about 20:1, from about 0.1:1 to about 10:1, from about 0.2:1 to about 5:1, from about 0.5:1 to about 2:1, or any intermediate ranges or ratios thereof. In certain embodiments, the ratio between 2′FL and LNnT in the food composition is about 10:1, about 9:1, about 8:1, about 7:1, about 6:1, about 5:1, about 4:1, about 3:1, about 2:1, about 1.9:1, about 1.8:1, about 1.7:1, about 1.6:1, about 1.5:1, about 1.4:1, about 1.3:1, about 1.2:1, about 1.1:1, about 1:1, or any intermediate ratios thereof. In certain embodiments, the ratio between 2′FL and LNnT in the food composition is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10 or any intermediate ratios thereof. The ratio by weight between 2′FL and LNnT in the food composition is from about 7:1 to about 8:1

In certain embodiments, the food composition comprises an oligosaccharide consisting of 2 galactose moieties and a hexose moiety. In certain embodiments, the galactose moieties are covalently linked to each other. In certain embodiments, at least one of the galactose moieties is in α-galactose form. In certain embodiments, at least one of the galactose moieties is in β-galactose form. In certain embodiments, the hexose moiety is a glucose moiety.

### 3. Methods of Use

In certain non-limiting embodiments, the presently disclosed subject matter provides for a method for improving health of a companion animal in need thereof. In certain embodiments, the method can improve immunity, digestive function and/or decrease inflammation of a companion animal. In certain non-limiting embodiments, the presently disclosed subject matter provides for a method for modifying a gastrointestinal (GI) microbiome in a companion animal. In certain non-limiting embodiments, the presently disclosed subject matter provides for a method for maintaining and/or increasing a probiotic in a companion animal. In certain embodiments, the method comprises administering an effective amount of a companion animal food product comprising any oligosaccharide disclosed herein. In certain embodiments, the oligosaccharide can be consumed by a probiotic. In certain embodiments, the companion animal food product further comprises a probiotic.

In certain non-limiting embodiments, the companion animal is a feline (e.g., a domestic cat) or a canine (e.g., a domestic dog).

In certain non-limiting embodiments, the companion animal is at risk of a digestive disorder and/or inflammation. In certain non-limiting embodiments, the companion animal is not known to be at risk of a digestive disorder and/or inflammation. In certain non-limiting embodiments, the companion animal has a digestive disorder and/or inflammation. In certain non-limiting embodiments, the companion animal is not known to have a digestive disorder and/or inflammation. In certain non-limiting embodiments, the companion animal is under a treatment for a digestive disorder and/or inflammation. In certain non-limiting embodiments, the treatment is a dietary therapy. In certain embodiments, the digestive disorder can be irritable bowel syndrome, constipation, gastritis, colitis, inflammatory bowel disease, gastrointestinal ulcers, haemorrhagic gastroenteritis, diarrhea, antibiotic associated diarrhea, acute or chronic enteropathy, and necrotizing enterocoloitis.

In certain embodiments, the probiotic is a feline probiotic. In certain embodiments, the animal probiotic is a canine probiotic. In certain embodiments, the probiotic is bifidobacterium lactic acid bacterium and/or enterococcus. In certain embodiments, the probiotic is selected from the group consisting of any organism from lactic acid bacteria and more specifically from the following bacterial genera; Lactococcus spp., Pediococcus spp., Bifidobacterium spp. (e.g., B. longum B. bifidum, B. pseudolongum, B. animalis), Lactobacillus spp. (e.g. L. bulgaricus, L. acidophilus, L. brevis, L casei, L. rhamnosus, L. plantarum, L. reuteri, L. fermentum, Enterococcus spp. (e.g. E. faecium), Prevotella spp., Fusobacteria spp, Alloprevotella spp, Bacteroides spp., and any combination thereof.

In certain embodiments, the population of the probiotic can be increased by at least about 5%, at least about 10%, at least about 20%, at least about 30%, at least about 40%, at least about 50%, at least about 100%, at least about 200%, at least about 300%, at least about 500%, at least about 1000% after the treatment. In certain embodiments, the population of the probiotic can be increased by at least about 20-fold, at least about 50-fold, at least about 100-fold, at least about 500-fold, at least about 1000-fold, at least about 5000-fold, at least about 10000-fold or more after the treatment. In certain embodiments, the population of the probiotic can be measured within about 1 day, about 2 days, about 3 days, about 4 days, about 5 days, about 6 days, about 1 week, about 2 weeks, about 3 weeks, about 4 weeks, about one month, about two months or about three months after the initiation of the treatment.

In certain embodiments, the companion animal is less than about 1 year old, less than about 11 months old, less than about 10 months old, less than about 9 months old, less than about 8 months old, less than about 7 months old, less than about 6 months old, less than about 5 months old, less than about 4 months old, less than about 3 months old, less than about 2 months old, or less than about 1 month old. In certain embodiments, the companion animal is less than about 10 weeks old, less than about 9 weeks old, less than about 8 weeks old, less than about 7 weeks old, less than about 6 weeks old, less than about 5 weeks old, less than about 4 weeks old, less than about 3 weeks old, less than about 2 weeks old, or less than about 1 week old. In certain embodiments, the companion animal is less than about 10 days old, less than about 9 days old, less than about 8 days old, less than about 7 days old, less than about 6 days old, less than about 5 days old, less than about 4 days old, less than about 3 days old, less than about 2 days old, or less than about 1 day old.

In certain embodiments, the companion animal is between about 1 day old and about 10 weeks old, between about 2 day old and about 9 weeks old, between about 3 day old and about 8 weeks old, between about 4 day old and about 7 weeks old, between about 4 day old and about 6 weeks old, between about 1 day old and about 5 weeks old, between about 1 day old and about 4 weeks old, between about 1 day old and about 3 weeks old, between about 1 day old and about 2 weeks old, or between about 1 day old and about 1 week old.

In certain embodiments, the companion animal is more than about 1 day old, more than about 2 days old, more than about 3 days old, more than about 4 days old, more than about 5 days old, more than about 6 days old, more than about 1 week old, more than about 2 weeks old, more than about 3 weeks old, more than about 4 weeks old, more than about 1 month old, more than about 2 months old, more than about 3 months old, more than about 4 months old, more than about 5 months old, more than about 6 months old, more than about 1 year old, more than about 2 years old, more than about 3 years old, more than about 4 years old, or more than about 5 years old.

In certain embodiments, the companion animal is between about 1 month old and about 1 year old, between about 1 year old and about 2 years old, between about 2 years old and about 3 years old, between about 3 years old and about 4 years old, between about 4 years old and about 5 years old, between about 5 years old and about 10 years old, or between about 10 years old and about 15 years old.

In certain embodiments, the companion animal is a canine and is less than about 38 days old. In certain embodiments, the companion animal is a feline and is less than about 36 days old. In certain embodiments, the companion animal is less than about 10 days old. In certain embodiments, the companion animal is less than about 4 days old.

In certain embodiments, the companion animal food product can be fed to a companion animal from 20 times per day to once per day, from 10 times per day to once per day, or from 5 times per day to once per day. In certain embodiments, the companion animal food product can be fed to a companion animal once per day, twice per day, thrice per day, 4 times per day, 5 times per day, 6 times per day, 7 times per day, 8 times per day, 9 times per day, 10 or more times per day. In certain embodiments, the companion animal food product can be fed to a companion animal once per two days, once per three days, once per four days, once per five days, once per six days, once a week, once per two weeks, once per three weeks, or once per month. In certain embodiments, the companion animal food product can be fed to an animal in a constant manner, e.g., where the animal grazes on a constantly available supply of the companion animal food product.

In certain embodiments, the dosage of the oligosaccharide is between about 1 mg/kg body weight per day and about 5000 mg/kg body weight per day. In certain embodiments, the dosage of the oligosaccharide is between about 5 mg/kg body weight per day and about 1000 mg/kg body weight per day, between about 10 mg/kg body weight per day and about 500 mg/kg body weight per day, between about 10 mg/kg body weight per day and about 250 mg/kg body weight per day, between about 10 mg/kg body weight per day and about 200 mg/kg body weight per day, between about 20 mg/kg body weight per day and about 100 mg/kg body weight per day, between about 20 mg/kg body weight per day and about 50 mg/kg body weight per day or any intermediate range thereof. In certain embodiments, the dosage of the oligosaccharide is at least about 1 mg/kg body weight per day, at least about 5 mg/kg body weight per day, at least about 10 mg/kg body weight per day, at least about 20 mg/kg body weight per day, at least about 50 mg/kg body weight per day, at least about 100 mg/kg body weight per day, at least about 200 mg/kg body weight per day or more. In certain embodiments, the dosage of the oligosaccharide is no more than about 5 mg/kg body weight per day, no more than about 10 mg/kg body weight per day, no more than about 20 mg/kg body weight per day, no more than about 50 mg/kg body weight per day, no more than about 100 mg/kg body weight per day, no more than about 200 mg/kg body weight per day, no more than about 500 mg/kg body weight per day or more.

In certain embodiments, the concentration of at least one oligosaccharide decreases over the course of feeding a companion animal. In certain embodiments, the concentration of at least one oligosaccharide increases over the course of feeding a companion animal. In certain embodiments, the concentration of at least one oligosaccharide is modified based on the age of the companion animal.

### 4. End Products

The presently disclosed subject matter provides a food product comprising one or more oligosaccharides disclosed herein. In certain embodiments, the food product comprises at least two oligosaccharides disclosed herein. In certain embodiments, the food product comprises at least three oligosaccharides. In certain embodiments, the food product comprises at least four, five, six, seven, eight or more oligosaccharides.

In certain embodiments, the oligosaccharide in the companion animal food product is at an amount of about 0.1 mg to about 5000 mg per daily serving or unit dosage. For example, and not by way of limitation, the oligosaccharide can be at an amount of about 1 mg to about 1000 mg per daily serving or unit dosage, about 10 mg to about 500 mg per daily serving or unit dosage, about 10 mg to about 300 mg per daily serving or unit dosage, about 10 mg to about 200 mg per daily serving or unit dosage, about 20 mg to about 200 mg per daily serving or unit dosage, about 30 mg to about 200 mg per daily serving or unit dosage, about 40 mg to about 200 mg per daily serving or unit dosage, about 50 mg to about 200 mg per daily serving or unit dosage, about 50 mg to about 150 mg per daily serving or unit dosage, about 50 mg to about 140 mg per daily serving or unit dosage, about 50 mg to about 130 mg per daily serving or unit dosage, about 50 mg to about 120 mg per daily serving or unit dosage, about 50 mg to about 100 mg per daily serving or unit dosage, about 20 mg to about 100 mg per daily serving or unit dosage, about 10 mg to about 50 mg per daily serving or unit dosage, about 100 mg to about 150 mg per daily serving or unit dosage, about 150 mg to about 200 mg per daily serving or unit dosage, about 100 mg to about 200 mg per daily serving or unit dosage, about 100 mg to about 300 mg per daily serving or unit dosage, about 100 mg to about 400 mg per daily serving or unit dosage, about 100 mg to about 500 mg per daily serving or unit dosage, about 200 mg to about 500 mg per daily serving or unit dosage, about 300 mg to about 500 mg per daily serving or unit dosage, about 200 mg to about 800 mg per daily serving or unit dosage or about 500 mg to about 1000 mg per daily serving or unit dosage. In certain embodiments, the oligosaccharide in the companion animal food product is at an amount of about 10 mg to about 200 mg per daily serving or unit dosage, e.g., about 50 mg per daily serving or unit dosage.

In certain embodiments, the oligosaccharide in the companion animal food product is at an amount of about 0.001% to about 10% weight by weight of the companion animal food product. For example, and not by way of limitation, the oligosaccharide can be at an amount of about 0.001% to about 0.01% weight by weight, about 0.01% to about 0.1% weight by weight, about 0.1% to about 1% weight by weight, about 1% to about 10% weight by weight, about 0.01% to about 0.5% weight by weight, about 0.01% to about 0.25% weight by weight, about 0.01% to about 0.2% weight by weight, about 0.01% to about 0.15% weight by weight, about 0.01% to about 0.1% weight by weight, about 0.01% to about 0.05% weight by weight, about 0.1% to about 0.15% weight by weight, about 0.1% to about 0.2% weight by weight, about 0.1% to about 0.25% weight by weight, about 0.1% to about 0.5% weight by weight, about 0.5% to about 1% weight by weight, about 0.1% to about 2% weight by weight, about 0.1% to about 5% weight by weight, about 0.1% to about 10% weight by weight, about 2% to about 10% weight by weight, and about 5% to about 10% weight by weight. In certain embodiments, the oligosaccharide in the companion animal food product is at an amount of no more than about 0.5% weight by weight. In certain embodiments, the oligosaccharide in the companion animal food product is at an amount of no more than about 0.2% weight by weight. In certain embodiments, the oligosaccharide in the companion animal food product is at an amount of no more than about 0.15% weight by weight. In certain embodiments, the oligosaccharide in the companion animal food product is at an amount of no more than about 0.1% weight by weight. In certain embodiments, the oligosaccharide in the companion animal food product is at an amount of no more than about 0.05% weight by weight.

In certain embodiments, the oligosaccharide in the companion animal food product is at a total concentration of about 0.001 g/L to about 100 g/L of the companion animal food product. For example, and not by way of limitation, the oligosaccharide can be at a total concentration of about 0.01 g/L to about 0.1 g/L, about 0.1 g/L to about 1 g/L, about 1 g/L to about 10 g/L, about 10 g/L to about 100 g/L, about 0.1 g/L to about 5 g/L, about 0.1 g/L to about 2.5 g/L, about 0.1 g/L to about 2 g/L, about 0.1 g/L to about 1.5 g/L, about 0.1 g/L to about 1 g/L, about 0.1 g/L to about 0.5 g/L, about 1 g/L to about 1.5 g/L, about 1 g/L to about 2 g/L, about 1 g/L to about 2.5 g/L, about 1 g/L to about 5 g/L, about 5 g/L to about 10 g/L, about 1 g/L to about 20 g/L, about 1 g/L to about 50 g/L, about 0.1 g/L to about 10 g/L, about 0.2 g/L to about 10 g/L, and about 0.5 g/L to about 10 g/L. In certain embodiments, the oligosaccharide in the companion animal food product is at a total concentration of no more than about 5 g/L. In certain embodiments, the oligosaccharide in the companion animal food product is at a total concentration of no more than about 2 g/L. In certain embodiments, the oligosaccharide in the companion animal food product is at a total concentration of no more than about 1.5 g/L. In certain embodiments, the oligosaccharide in the companion animal food product is at a total concentration of no more than about 1 g/L. In certain embodiments, the oligosaccharide in the companion animal food product is at a total concentration of no more than about 0.5 g/L. In certain embodiments, the oligosaccharide in the companion animal food product is at a total concentration of about 0.3 g/L to about 5.0 g/L, about 1.2 g/L to about 2.2 g/L, about 0.5 g/L to about 1.5 g/L, about 0.01 g/L to about 1.5 g/L of the companion animal food product.

In certain embodiments, a formulation of the presently disclosed subject matter can further comprise an additional active agent. Non-limiting examples of additional active agents that can be present within a formulation of the presently disclosed subject matter include a nutritional agent (e.g., amino acids, peptides, proteins, fatty acids, carbohydrates, sugars, nucleic acids, nucleotides, vitamins, minerals, etc.), a prebiotic, a probiotic, an antioxidant, and/or an agent that improves animal health.

In certain embodiments, the food product comprises one or more animal probiotic. In certain embodiments, at least one of the animal probiotic can feed on at least one oligosaccharide comprised in the food product. In certain embodiments, the animal probiotic is a feline probiotic. In certain embodiments, the animal probiotic is a canine probiotic. In certain embodiments, the probiotic is bifidobacterium, lactic acid bacterium and/or enterococcus. In certain embodiments, the probiotic is selected from the group consisting of any organism from lactic acid bacteria and more specifically from the following bacterial genera; Lactococcus spp., Pediococcus spp., Bifidobacterium spp. (e.g., B. longum B. bifidum, B. pseudolongum, B. animalis), Lactobacillus spp. (e.g. L. bulgaricus, L. acidophilus, L. brevis, L casei, L. rhamnosus, L. plantarum, L. reuteri, L. fermentum, Enterococcus spp. (e.g. E. faecium), Prevotella spp., Fusobacteria spp, Alloprevotella spp, and any combination thereof. In certain embodiments, the probiotic is administered to a companion animal in an amount of from about 1 colony forming unit (CFU) to about 100 billion CFUs per day for the maintenance of GI microflora. In certain embodiments, the probiotic is administered to a companion animal in an amount of from about 1 colony forming unit (CFU) to about 20 billion CFUs per day for the maintenance of GI microflora. In certain embodiments, the probiotic is administered to a companion animal in an amount of from about 1 billion CFUs to about 20 billion CFUs per day for the maintenance of GI microflora. In certain embodiments, the probiotic is administered to a companion animal in amounts of from about 0.01 billion to about 100 billion live bacteria per day. In certain embodiments, the probiotic is administered to a companion animal in amounts of from about 0.1 billion to about 10 billion live bacteria per day.

In certain embodiments, an additional prebiotic can be included, such as fructooligosaccharides (FOS), xylooligosaccharides (XOS), galactooligosaccharides (GOS), glucans, galactans, arabinogalactan, inulin and/or mannooligosaccharides. In certain embodiments, the additional prebiotic is administered in amounts sufficient to positively stimulate the GI microflora and/or cause one or more probiotics to proliferate.

In certain embodiments, the companion animal food product can further contain additives known in the art. In certain embodiments, such additives are present in amounts that do not impair the purpose and effect provided by the presently disclosed subject matter. Examples of contemplated additives include, but are not limited to, substances that are functionally beneficial to improving health, substances with a stabilizing effect, organoleptic substances, processing aids, substances that enhance palatability, coloring substances, and substances that provide nutritional benefits. In certain embodiments, the stabilizing substances include, but are not limited to, substances that tend to increase the shelf life of the product. In certain embodiments, such substances include, but are not limited to, preservatives, synergists and sequestrants, packaging gases, stabilizers, emulsifiers, thickeners, gelling agents, and humectants. In certain embodiments, the emulsifiers and/or thickening agents include, for example, gelatin, cellulose ethers, starch, starch esters, starch ethers, and modified starches.

In certain embodiments, the additives for coloring, palatability, and nutritional purposes include, for example, colorants; iron oxide, sodium chloride, potassium citrate, potassium chloride, and other edible salts; vitamins; minerals; and flavoring. The amount of such additives in a product typically is up to about 5% (dry basis of the product).

In certain embodiments, the companion animal food product is a dietary supplement. In certain embodiments, the dietary supplements include, for example, a feed used with another feed to improve the nutritive balance or performance of the total. In certain embodiments, the supplements include compositions that are fed undiluted as a supplement to other feeds, offered free choice with other parts of an animal's ration that are separately available, or diluted and mixed with an animal's regular feed to produce a complete feed. The AAFCO, for example, provides a discussion relating to supplements in the American Feed Control Officials, Incorp. Official Publication, p. 220 (2003). Supplements can be in various forms including, for example, powders, liquids, syrups, pills, tablets, encapsulated compositions, etc.

In certain embodiments, the companion animal food product is a treat. In certain embodiments, treats include, for example, compositions that are given to an animal to entice the animal to eat during a non-meal time. In certain embodiments, the companion animal food product is a treat for canines include, for example, dog bones. Treats can be nutritional, wherein the product comprises one or more nutrients, and can, for example, have a composition as described above for food. Non-nutritional treats encompass any other treats that are non-toxic.

In certain embodiments, oligosaccharides of the presently disclosed subject matter can be incorporated into the composition during the processing of the formulation, such as during and/or after mixing of other components of the product. Distribution of these components into the product can be accomplished by conventional means.

In certain embodiments, companion animal food products of the presently disclosed subject matter can be prepared in a canned or wet form using conventional companion animal food processes. In certain embodiments, ground animal (e.g., mammal, poultry, and/or fish) proteinaceous tissues are mixed with the other ingredients, such as milk fish oils, cereal grains, other nutritionally balancing ingredients, special purpose additives (e.g., vitamin and mineral mixtures, inorganic salts, cellulose and beet pulp, bulking agents, and the like); and water that sufficient for processing is also added. These ingredients are mixed in a vessel suitable for heating while blending the components. Heating of the mixture can be effected using any suitable manner, such as, for example, by direct steam injection or by using a vessel fitted with a heat exchanger. Following the addition of the last ingredient, the mixture is heated to a temperature range of from about 50° F. to about 212° F. Temperatures outside this range are acceptable but can be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. This is usually accomplished by heating to temperatures of greater than about 230° F. for an appropriate time, which is dependent on, for example, the temperature used and the composition.

In certain embodiments, companion animal food products of the presently disclosed subject matter can be prepared in a dry form using conventional processes. In certain embodiments, dry ingredients, including, for example, animal protein sources, plant protein sources, grains, etc., are ground and mixed together. In certain embodiments, moist or liquid ingredients, including fats, oils, animal protein sources, water, etc., are then added to and mixed with the dry mix. In certain embodiments, the mixture is then processed into kibbles or similar dry pieces. In certain embodiments, the companion animal food product is kibble. In certain embodiments, kibble is formed using an extrusion process in which the mixture of dry and wet ingredients is subjected to mechanical work at a high pressure and temperature and forced through small openings and cut off into kibble by a rotating knife. In certain embodiments, the wet kibble is then dried and optionally coated with one or more topical coatings which can include, for example, flavors, fats, oils, powders, and the like. In certain embodiments, kibble can also be made from the dough using a baking process, rather than extrusion, wherein the dough is placed into a mold before dry-heat processing.

In certain embodiments, treats of the presently disclosed subject matter can be prepared by, for example, an extrusion or baking process similar to those described above for dry food.

### EXAMPLES

The presently disclosed subject matter will be better understood by reference to the following Example, which is provided as exemplary of the invention, and not by way of limitation.

### Example 1

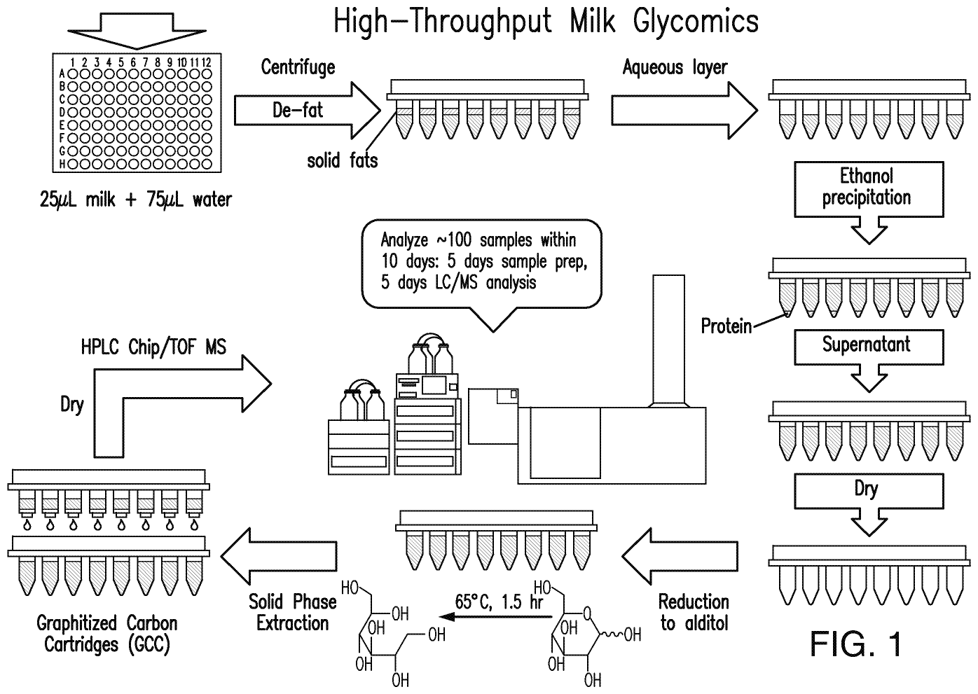

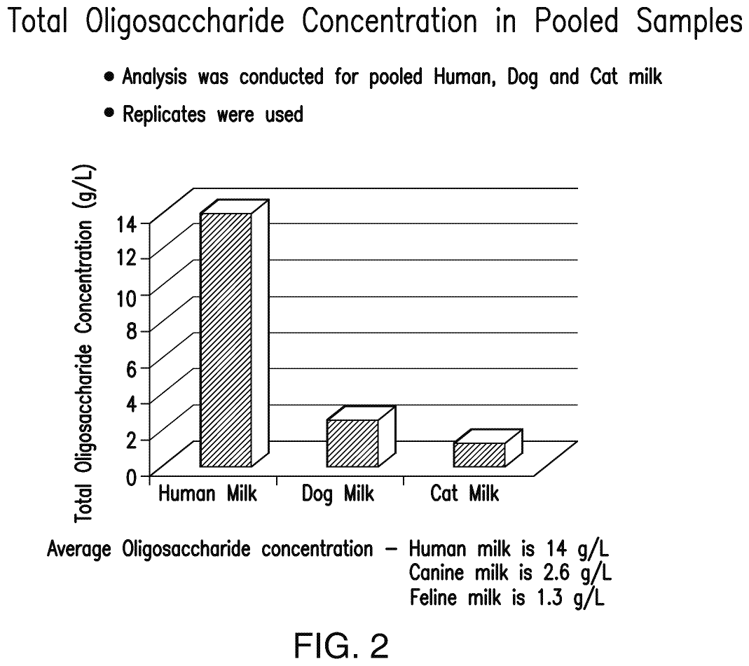

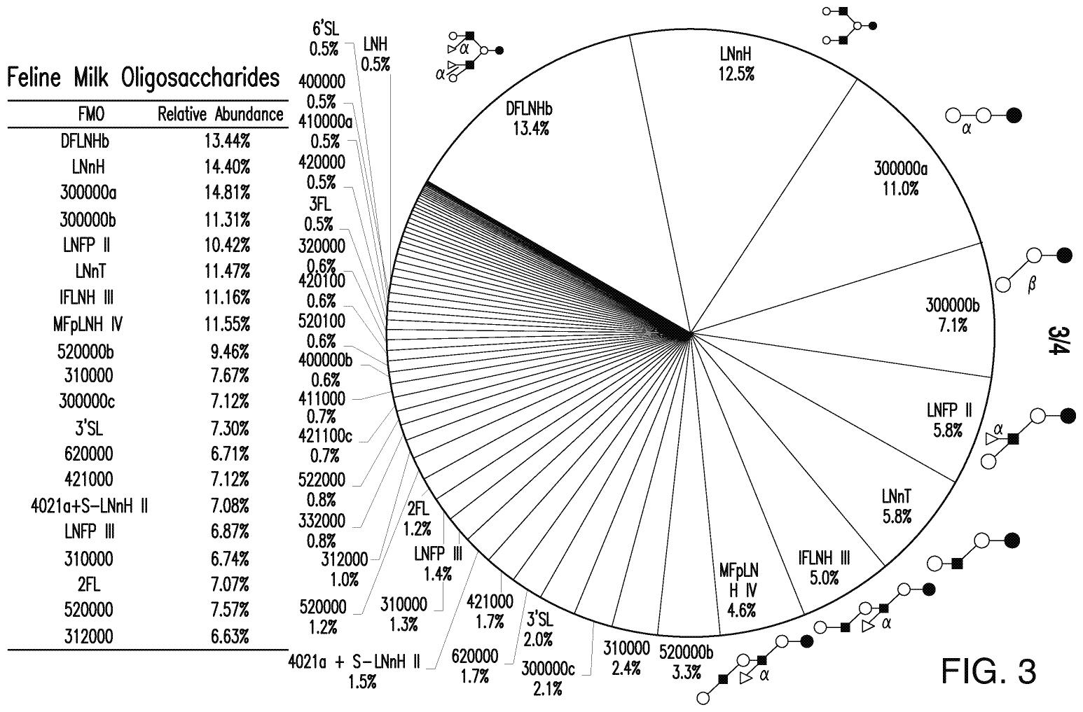

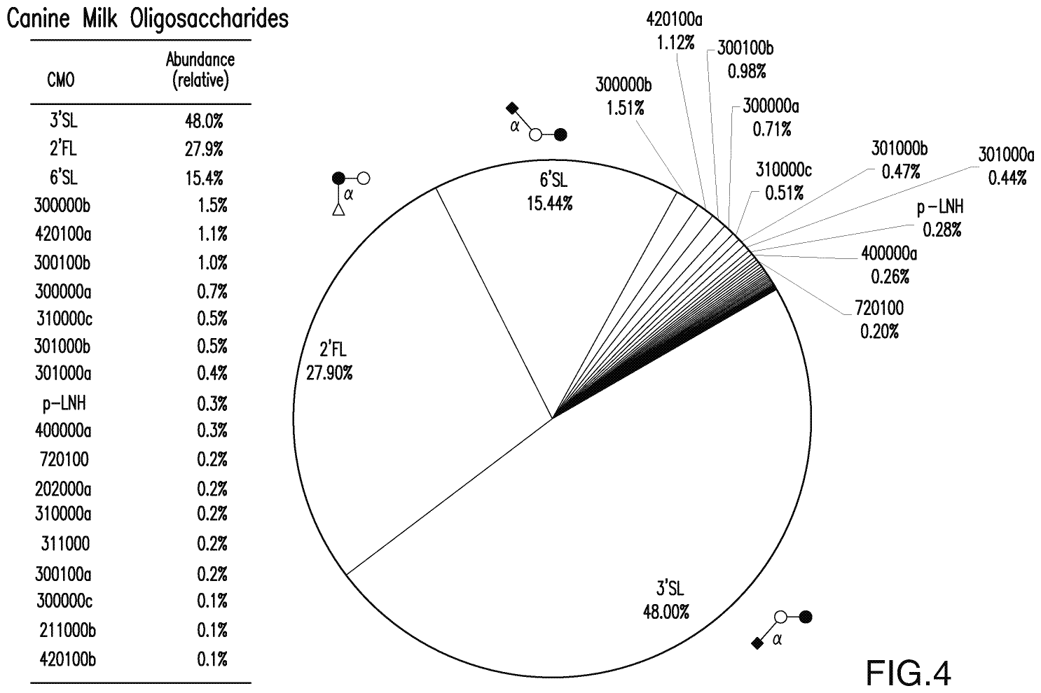

The oligosaccharide profiles of canine and feline milk were determined using high-throughput milk glycomics of milks from various breeds of domestic dogs and cats. A schematic representation of the process for purifying and detecting milk oligosaccharides is shown in FIG. 1. Canine and feline milk had lower concentrations of total oligosaccharides compared to human milk, as shown in FIG. 2. The oligosaccharides of feline milk and canine milk were profiled, and the results are shown in FIGS. 3 and 4, respectively.

### Example 2

A companion animal food product is made comprising oligosaccharides DFLNHb, 3′SL and LNnH in a total concentration of from about 0.30 to about 5.0 g/L, wherein DFLNHb, 3′SL and LNnH are at a ratio by weight of 0.8 to 0.6 to 0.5.

Cats of less than 36 days old, less than 10 days old, and less than 4 days old are fed with this food product.

A companion animal food product is made comprising oligosaccharides 3′SL, LNnT, 2′FL and LNT in a total concentration of from about 0.5 g/L to about 1.5 g/L, wherein 3′SL, LNnT, 2′FL and LNT are at a ratio by weight of 0.6 to 0.3 to 0.06 to 0.01.

Cats of less than 36 days old, less than 10 days old, and less than 4 days old are fed with this food product. Dogs of less than 38 days old, less than 10 days old, and less than 4 days old are fed with this food product.

A companion animal food product is made comprising 3′SL, 2′FL, 6′SL, and LNnT in a total concentration of from about 0.01 g/L to about 1.5 g/L, wherein 3′SL, 2′FL, 6′SL, and LNnT are at a ratio by weight of 0.46 to 0.23 to 0.17 to 0.004.

Dogs of less than 38 days old, less than 10 days old, and less than 4 days old are fed with this food product.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A companion animal food product comprising oligosaccharides difucosyllacto-N-hexaose b (DFLNHb) and lacto-N-neohexaose (LNnH).

2. The companion animal food product of claim 1 further comprising 3′-sialyllactose (3′SL).

3. The companion animal food product of claim 1, further comprising a oligosaccharide selected from the group consisting of 3′-sialyllactose (3′SL), 2′-fucosyllactose (2′FL), 6′-sialyllactose (6′SL), lacto-N-neotetraose (LNnT), lacto-N-tetraose (LNT), lacto-N-fucopentaose II (LNFP II), fucosyl-paralacto-N-hexaose III (IFLNH III), monofucosyl-paralacto-N-hexaose IV (MFpLNH IV), lacto-N-hexaose (LNH), sialyl-lacto-N-neohexaose II (S-LNnH II), para-lacto-N-hexaose (pLNH), 3′-fusosyllactose (3′FL), difucosyl-para-lacto-N-Hexoase II (DFpLNH II), lacto-difucosyl-tetraose (LDFT), and any combination thereof.

4. (canceled)

5. The companion animal food product of claim 1, wherein a total concentration of the oligosaccharides is from about 0.3 g/L to about 5.0 g/L in a final food product.

6. A companion animal food product comprising oligosaccharides 2′-fucosyllactose (2′FL) and lacto-N-neotetraose (LNnT).

7. The companion animal food product of claim 6, further comprising a oligosaccharide selected from the group consisting of difucosyllacto-N-hexaose b (DFLNHb), lacto-N-neohexaose (LNnH), 3′-sialyllactose (3′SL), 6′-sialyllactose (6′SL), lacto-N-tetraose (LNT), lacto-N-fucopentaose II (LNFP II), fucosyl-paralacto-N-hexaose III (IFLNH III), monofucosyl-paralacto-N-hexaose IV (MFpLNH IV), lacto-N-hexaose (LNH), sialyl-lacto-N-neohexaose II (S-LNnH II), 3′-fusosyllactose (3′FL), difucosyl-para-lacto-N-hexoase II (DFpLNH II), lacto-difucosyl-tetraose (LDFT), para-lacto-N-hexaose (pLNH) and any combination thereof.

8. A companion animal food product comprising oligosaccharides 3′-sialyllactose (3′SL), 2′-fucosyllactose (2′FL) and 6′-sialyllactose (6′SL), wherein the companion animal is a canine and wherein a total concentration of the oligosaccharides is from about 0.3 g/L to about 5.0 g/L in a final food product.

9. The companion animal food product of claim 8, wherein a total concentration of the oligosaccharides is from about 1.2 g/L to about 2.2 g/L in a final food product.

10.-18. (canceled)

19. The companion animal food product of claim 1, wherein the companion animal food product further comprises a probiotic.

20.-25. (canceled)

26. The companion animal food product of claim 19, wherein the probiotic is selected from the group consisting of any microorganism from Lactococcus spp., Pediococcus spp., Bifidobacterium spp., Lactobacillus spp., Enterococcus spp., B. longum, B. bifidum, B. pseudolongum, B. animalis, L. bulgaricus, L. acidophilus, L. brevis, L casei, L. rhamnosus, L. plantarum, L. reuteri, L. fermentum, E. faecium and any combination thereof.

27.-55. (canceled)

56. The companion animal food product of claim 2 further comprising 2′-fucosyllactose (2′-FL) and lacto-N-neotetraose (LNnT).

57. The companion animal food product of claim 56, wherein a total concentration of the oligosaccharides is from about 0.3 g/L to about 5.0 g/L in a final food product.

58. The companion animal food product of claim 2, wherein a total concentration of the oligosaccharides is from about 0.3 g/L to about 5.0 g/L in a final food product.

59. The companion animal food product of claim 2, wherein the DFLNHb, 3′SL and LNnH are in a ratio by weight of about 8 to about 6 to about 5, respectively.

60. The companion animal food product of claim 6, further comprising 3′-sialyllactose (3′SL) and lacto-N-tetraose (LNT).

61. The companion animal food product of claim 60, wherein a total concentration of the oligosaccharides is from about 0.3 g/L to about 5.0 g/L in a final food product.

62. The companion animal food product of claim 61, wherein the 3′SL, LNnT, 2′FL and LNT are in a ratio by weight of about 6 to about 3 to about 0.6 to about 0.1, respectively.

63. The companion animal food product of claim 6, further comprising 3′-sialyllactose (3′SL) and 6′-sialyllactose (6′SL).

64. The companion animal food product of claim 63, wherein a total concentration of the oligosaccharides is from about 0.3 g/L to about 5.0 g/L in a final food product.

65. The companion animal food product of claim 64, wherein the 3′SL, 2′FL, 6′SL and LNnT is in a ratio by weight of about 46 to about 23 to about 17 to about 0.4, respectively.
